(19) United States
(12) Patent Application Publication
(10) Pub. No.: US 2006/0254578 A1
(43) Pub. Date: Nov. 16, 2006

(54) Inhalation therapy device with a nebulising nozzle and a compressor

(75) Inventors: Andreas Boehm, Reichling (DE); Eduard Kunschir, Munich (DE)

Correspondence Address: WOLF GREENFIELD & SACKS, PC, FEDERAL RESERVE PLAZA, 600 ATLANTIC AVENUE, BOSTON, MA, 02210-2206, US

(73) Assignee: Pari GmbH Spezialisten fuer effektive Inhalation, Starnberg (DE)

(21) Appl. No.: 11/431,350

(22) Filed: May 10, 2006

(30) Foreign Application Priority Data
May 13, 2005 (DE) DE102005022339.7

(51) Int. Cl. A61M 11/00

(52) U.S. Cl. 128/200.14

(57) ABSTRACT

The inhalation therapy device comprises a nebulising nozzle 1, to which compressed air 6 can be supplied for nebulising a liquid 5, a compressor 8 having an electromotive drive 8b for generating the compressed air supplied to the nebulising nozzle, and an electric energy store 9 for providing electric energy for the electromotive drive 8b of the compressor 8. The electric energy store 9 thereby comprises a stabilising device 9b that ensures a constant output voltage of the energy store 9.

The invention relates to inhalation therapy devices having a nebulising nozzle for nebulising a liquid by means of compressed air, and having a compressor for providing the compressed air to be supplied to the nebulising nozzle.

In addition to other primary aerosol generators, nebulising nozzles, which generate an aerosol from a liquid by means of supplied compressed air, have particularly proven themselves to be of value for the generation of an aerosol for carrying out an inhalation therapy. Nebulising nozzles are known, for example, from DE 27 11 060 A, DE 89 05 364 U or DE 196 02 628 A. The compressed air for aerosol generation is provided by means of a compressor that generally comprises an electromotor as the drive mechanism. In portable inhalation therapy devices, such as described, for example, in DE 94 18 334 U, the compressor is operated by means of an accumulator as the electric energy store.

Known from the prior art are various rechargeable electric energy stores which can be used in a portable inhalation therapy device. Unlike electric energy stores for electronic devices, for example in the field of mobile information and communication systems, it is not to be assumed in the case of portable inhalation therapy devices comprising a nebulising nozzle that an electric energy store having particular properties should be used for operation of the compressor. A number of accumulator systems are basically suitable for this case of use. It may be known in the prior art to use, in the field of mobile information and communication systems, electric energy stores in which a constant output voltage is ensured by means of a regulator, as is described, for example, in DE 101 38 515 A. However, the application environment in inhalation therapy devices having a nebulising nozzle and a compressor considerably differs therefrom since the compressor must be regarded as an electric unit that places only very low demands on the voltage supply, which is completely different to, for example, a device from the field of mobile information and communication technology.

In view of the above, the present invention proposes to equip an inhalation therapy device having a nebulising nozzle and a compressor with an electric energy store (accumulator/battery pack), in which, by means of a regulator or voltage converter, for example a DC/DC converter, a constant supply voltage is supplied for operation of the compressor.

An inhalation therapy device according to the invention is not only advantageous in view of the electric components, i.e. in particular the electromotor of the compressor, but also has an advantageous effect with regard to the inhalation therapy carried out with the inhalation therapy device according to the invention. This is because the provision of a constant supply voltage for operating the compressor of a portable inhalation therapy device ensures that the entire system is improved as regards the amount released (TOR=total output rate) and the quality (MMD=mass median diameter) of the aerosol and that more constant values are achieved. As a result, the dosage accuracy that can be attained with an inhalation therapy device according to the invention is improved.

An inhalation therapy device according to the invention comprises a nebulising nozzle, to which compressed air can be supplied for nebulising a liquid, a compressor having an electromotive drive for generating the compressed air supplied to the nebulising nozzle, and an electric energy store for providing electric energy for the electromotive drive of the compressor. The electric energy store thereby comprises a stabilising device that ensures a constant output voltage of the energy store. An accumulator is thus available for the inhalation therapy device, which supplies a constant output voltage and thus a defined maximum power.

The stabilising device can, for example, be realised in the form of a DC/DC converter. This virtually rules out any danger to the accumulator owing to a short-circuit or total discharge.

In an advantageous embodiment, the electric energy store comprises a housing in which the stabilising device is accommodated. An accumulator that can be handled as a single unit is therefore available.

In order to obtain an overall compact system, the shape and size of the housing of the electric energy store is adapted, in an advantageous design, to the housing of the compressor.

The invention is described below by means of an embodiment.

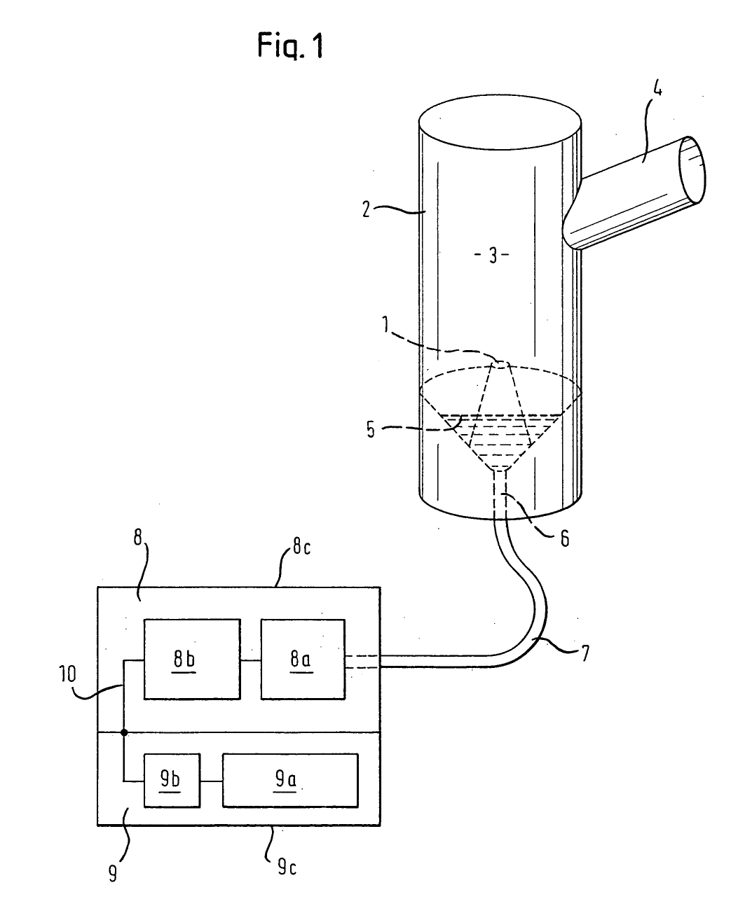

FIG. 1 shows an embodiment of an inhalation therapy device according to the invention.

The embodiment of an inhalation therapy device according to the invention as shown in FIG. 1 comprises a nebulising nozzle 1 which is disposed in a nebuliser housing 2 that surrounds a nebulising space 3. A patient inhales an aerosol generated by the nebulising nozzle 1 from the nebulising space 3 via a mouthpiece 4. In order to generate the aerosol, a liquid 5, which is advantageously stored in the nebuliser housing 2, and compressed air 6 are supplied to the nebulising nozzle 1.

The compressed air 6, which is preferably fed to the nebulising nozzle 1 via a hose line 7 or the like, is provided by a compressor 8. For this purpose, the compressor 8 typically comprises a compressed air generating means 8a, for example a piston pump, a diaphragm pump or a centrifugal pump, and an electromotive drive means 8b, preferably an electromotor.

Electric energy is supplied to the electromotive drive means 8b for operation by an electric energy store 9, which, according to the invention, comprises both a storage element 9a for electric energy and a device 9b for ensuring a constant output voltage. The electric energy is thus supplied at a constant voltage to the electromotive drive 8b of the compressor 8 via the supply line 10. A constant output voltage and thus a constant power is supplied over the entire discharge period of the storage element 9a. In the design according to the invention, it is not necessary to adapt the electric energy store to the connected load, for example by adapting the number of storage elements 9a.

In an advantageous embodiment, the stabilising device 9b is a DC/DC converter, to which voltages can be supplied in a predetermined area on the input side and which releases a constant output voltage on the output side.

In a further advantageous embodiment, the stabilising device 9b, as can be seen in FIG. 1, is accommodated in a housing 9c of the electric energy store 9. This results in a compact unit that is easy to handle. The housing 9c of the electric energy store 9 is preferably adapted to the shape and size of the housing of the compressor 8, which is also shown in FIG. 1.

1. An inhalation therapy device comprising a nebulising nozzles, to which compressed air can be supplied for nebulising a liquid, a compressor having an electromotive drive for generating the compressed air supplied to the nebulising nozzle, and an electric energy store for providing electric energy for the electromotive drive of the compressor, said electric energy store comprising a stabilising device that ensures a constant output voltage of the energy store.

2. An inhalation therapy device according to claim 1, wherein the stabilising device is a DC/DC converter.

3. An inhalation therapy device according to claim 1, wherein the electric energy store comprises a housing and the stabilising device is disposed in the housing of said electric energy store.

4. An inhalation therapy device according to claim 3, wherein the compressor comprises a housing and the housing of the electric energy store is adapted in terms of shape and size to the housing of said compressor.
